(19) United States
(12) Patent Application Publication
(10) Pub. No.: US 2026/0158181 A1
(43) Pub. Date: Jun. 11, 2026

(54) ULTRAVIOLET/INFRARED STERILIZATION DEVICE AND METHOD

(71) Applicant: LUMAEGIS, INC., Austin, TX (US)

(72) Inventors: Sudhir SUBRAMANYA, Austin, TX (US); John S. MORREALE, Austin, TX (US); Werner K. GOETZ, Austin, TX (US); Matthijs Hendrik KEUPER, Geldrop (NL)

(73) Assignee: LUMAEGIS, INC., Austin, TX (US)

(21) Appl. No.: 19/183,352

(22) Filed: Apr. 18, 2025

Related U.S. Application Data
(63) Continuation of application No. PCT/US2023/035650, filed on Oct. 21, 2023.
(60) Provisional application No. 63/418,214, filed on Oct. 21, 2022.

(51) Int. Cl. A61L 2/10; A61L 2/085; A61L 103/15

(52) U.S. Cl. CPC A61L 2/10; A61L 2/085; A61L 2103/15; A61L 2202/11; A61L 2202/122; A61L 2202/14

(57) ABSTRACT

A sterilizing device for sterilizing instruments includes a housing, a drawer assembly, a radiant system, a first transparent member and a control system. The drawer assembly is configured to slidably open relative to the housing and has a drawer rack supported by a drawer frame assembly. The radiant system includes a first light panel having a first plurality of ultraviolet-C (UV-C) light emitting diodes (LED's) and a first plurality of infrared radiation (IR) LED's. The first plurality of UV-C LED's are configured to emit UV-C radiation. The first plurality of IR LED's configured to emit thermal radiation. The first transparent member and the drawer frame assembly define a sterilization chamber that receives the UV-C and thermal radiation emitted by the radiant system thereby sterilizing the instruments during a sterilizing process. The control system initiates the sterilizing process based upon a user input received at a user interface panel.

### CROSS REFERENCE TO RELATED APPLICATIONS

This application is a bypass continuation of International Application No. PCT/US2023/035650 filed Oct. 21, 2023, which claims priority to U.S. Provisional Patent Application No. 63/418,214 filed Oct. 21, 2022. Each of the above identified applications are hereby incorporated by reference in their entirety.

### FIELD

The present disclosure relates to sterilization devices and more particularly to sterilizing devices using a combination of UV-C and IR radiation, e.g., generated by LEDs to rapidly sterilize medical and surgical instruments.

### BACKGROUND

Sterilization is essential for ensuring that surfaces, such as on medical and surgical tools do not transmit infectious pathogens to patients. The Centers for Disease Control and Prevention defines three levels of mitigation for prevention of transmission of pathogens. The first level is cleaning to remove dirt from objects and surfaces. It is typically done manually or mechanically using soap and water. Thorough cleaning is essential before sterilization or disinfection because dirt that remains on the surfaces of instruments reduces their effectiveness. The second level is the disinfection process that eliminates pathogenic microorganisms, except bacterial spores, on inanimate objects. Finally, the third level is the sterilization process that inactivates all forms of microbial life including bacterial spores. Sterilization is the ultimate process and shouldn't be confused with disinfection which is not sporicidal. Multiple approaches exist in healthcare facilities to sterilize, e.g., by using pressurized steam, dry heat, Ethylene Oxide, Hydrogen Peroxide plasma, and liquid chemicals.

Steam sterilization in autoclaves (moist heat in the form of pressurized, saturated steam) is a widely used sterilization method in the healthcare industry. The heat that an autoclave delivers via pressurized steam kills bacteria and other microorganisms by causing the organisms' structural proteins and enzymes to denature in an irreversible way making them nonfunctional. However, there are multiple challenges with steam sterilization. The typical duration for the sterilization process could exceed more than hour. Heat can damage some materials over multiple cycles. It can melt acrylics, distort PVC, and corrode metal tools. A product that is wet after steam sterilization can be a source for recontamination. Also, steam sterilizers require de-ionized water and significant amounts of electricity to operate—all of which can add up over the life of the unit. The cost of water alone can reach significant amount of money annually. In steam sterilization, energy and equipment costs have risen and have become an increasing economic burden on oral and medical care. In addition, productivity requirements for clinics continue to increase since healthcare facilities desire to sterilize tools in the shortest possible time. There is a need for a novel device and method to sterilize surfaces, medical and dental instruments in a fast, efficient, thorough, and cost-effective manner.

To date, there are no published efforts to reliably use UV-C based devices to sterilize surgical instruments.

### SUMMARY

A sterilizing device for sterilizing instruments includes a housing, a drawer assembly, a radiant system, a first transparent member and a control system. The housing defines an internal volume. The drawer assembly is configured to slidably open relative to the housing. The drawer assembly has a drawer rack supported by a drawer frame assembly. The drawer rack is configured to receive the instruments. The radiant system includes a first light panel having a first plurality of ultraviolet-C (UV-C) light emitting diodes (LED's) and a first plurality of infrared radiation (IR) LED's arranged across a first printed circuit board. The first plurality of UV-C LED's are configured to emit UV-C radiation. The first plurality of IR LED's configured to emit thermal radiation. The first transparent member is disposed between the drawer frame assembly and the first light panel. The first transparent member and the drawer frame assembly define a sterilization chamber that receives the UV-C and thermal radiation emitted by the radiant system thereby sterilizing the instruments during a sterilizing process. The control system initiates the sterilizing process based upon a user input received at a user interface panel.

According to additional features, the radiant system further comprises a second light panel having a second plurality of UV-C LED's and a second plurality of IR LED's arranged across a second printed circuit board. The second plurality of UV-C LED's are configured to emit UV-C radiation. The second plurality of IR LED's configured to emit thermal radiation. The first light panel is arranged above the drawer rack and the second light panel is arranged below the drawer rack.

In other features, the sterilizing device further comprises a tray assembly having a tray and a cover. The tray is configured to hold the instruments thereon. The tray assembly is formed of fluorinated ethylene propylene (FEP) that is configured to pass UV-C and IR light emitted by the radiant system. The UV-C light has wavelengths between 200 nm and 400 nm. The IR light has wavelengths between 700 nm and 1000 nm.

According to additional features, the drawer rack is supported by a rack support frame. The drawer rack includes a corrugated bottom surface and outer rim, the outer rim having a forward mounting lip that is coupled to a first deflected mounting arm of the rack support frame and a rearward mounting lip that is coupled to a second deflected mounting arm of the rack support frame.

In other features, the sterilizing device includes a heat sink assembly having a first heat sink positioned against the first light panel and a second heat sink positioned against the second light panel. The sterilizing device can further include a sensor that senses a position of the drawer assembly, wherein the sensor communicates a verification signal to the control system indicative of the drawer assembly in a closed position, the control system initiating the sterilizing process based on receipt of the verification signal. The sterilizing device can include an optical device that is configured to capture an image of the contents on the drawer rack and communicate the image to the control system. The control system can determine an instrument type of the instruments on the drawer rack and determines criteria of the sterilizing process on the instrument type, the criteria including at least one of sterilization time and intensity of the emitted radiation from the LED's.

In other features, the control system is configured to receive signals from the user interface panel indicative of criteria of the sterilizing process. The criteria can include at least one of a sterilization time and intensity of the emitted radiation from the LED's.

A method for sterilizing an instrument in a sterilizing device includes sliding a drawer assembly out of a housing of the sterilizing device to an open position. Instruments to be sterilized are placed onto a drawer rack of the drawer assembly. The drawer assembly is slid back into the housing to a closed position. A preferred sterilizing process is determined at a control system of the sterilizing device. The preferred sterilizing process is initiated at the control system. The preferred sterilizing process includes activating a radiant system including a first light panel having a first plurality of ultraviolet-C (UV-C) light emitting diodes (LED's) and a first plurality of infrared radiation (IR) LED's arranged across a first printed circuit board. The first plurality of UV-C LED's emit UV-C radiation. The first plurality of IR LED's emit thermal radiation.

In additional features, initiating the preferred sterilizing process further includes activating, at the radiant system, a second light panel having a second plurality of UV-C LED's and a second plurality of IR LED's arranged across a second printed circuit board, the second plurality of UV-C LED's emitting UV-C radiation, the second plurality of IR LED's emitting thermal radiation.

In examples, emitting the UV-C light comprises emitting wavelengths between 200 nm and 400 nm. Emitting the IR light comprises emitting wavelengths between 700 nm and 1000 nm.

In additional features, placing instruments to be sterilized onto a drawer rack comprises placing instruments in a tray assembly formed of fluorinated ethylene propylene (FEP) that is configured to pass UV-C and IR light emitted by the radiant system. Determining a preferred sterilizing process can additionally include capturing, at an optical device disposed in the sterilizing device, an image of the instruments. The image is communicated to the control system. The control system determines a type of instruments in the sterilizing device based on the image. Criteria of the preferred sterilizing process is selected based on the type of instruments. The criteria includes at least one of sterilization time and intensity of the emitted radiation from the LED's. In other configurations, the selected sterilizing process is determined based on the process input by a user at the user interface panel.

### BRIEF DESCRIPTION OF THE DRAWINGS

FIG. 1 is a front perspective view of a sterilizing device constructed in accordance to one example of the present disclosure and shown with a drawer assembly in a closed position;

FIG. 2 is a rear perspective view of the sterilizing device of FIG. 1;

FIG. 3 is a sectional view of the sterilizing device taken along lines 3-3 of FIG. 1;

FIG. 4 is a front perspective view of the sectional view of FIG. 3;

FIG. 5A is a sectional view of the sterilizing device taken along lines 5A-5A of FIG. 1;

FIG. 5B is a detail view of a light panel of FIG. 5A;

FIG. 6 is an exploded view of a radiant system of the sterilization device shown opposite a tray and lid of a tray assembly according to examples of the present disclosure; and

FIG. 7 is a front perspective view of the sterilizing device shown with the drawer assembly in the open position.

### DESCRIPTION

The present disclosure provides an innovative sterilization device, which uses a combination of UV-C radiation, e.g., generated by UV-C LEDs, and thermal radiation, e.g., generated by IR LEDs, and thereby, increases the effectivity of eliminating pathogens from medical and surgical instruments or tools prior to their use. The sterilization device is configured to receive various tray assemblies or containers which are transparent to UV-C and IR radiation holding the tools. The container or containers can be extracted or removed from the sterilization device for loading, unloading and storing the instruments to be sterilized.

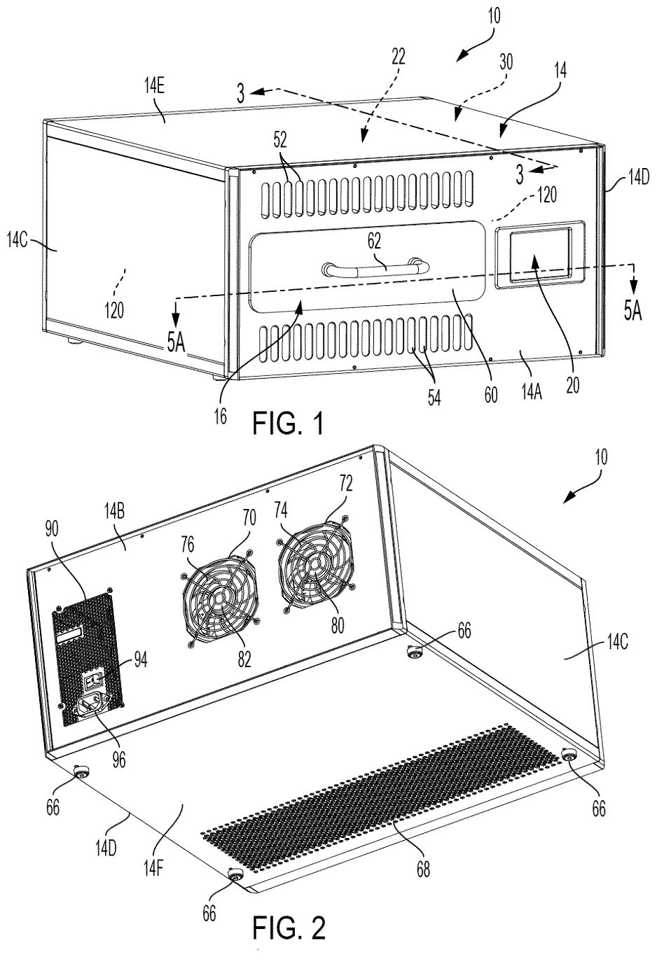

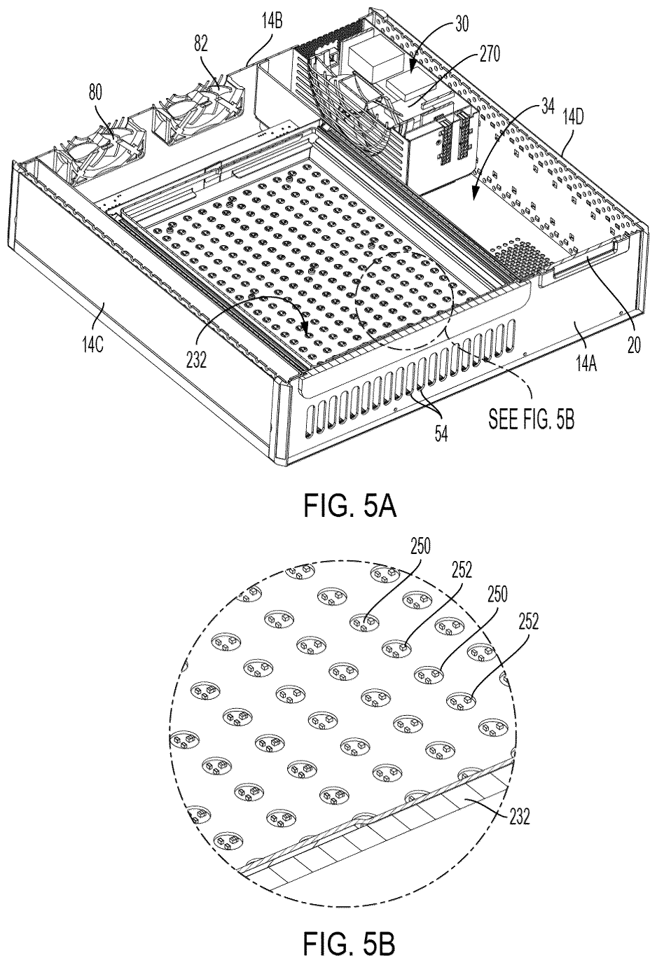

With initial reference to FIGS. 1-4 , a sterilizing device constructed in accordance to one example of the present disclosure is shown and generally identified at reference numeral 10. The sterilizing device 10 generally includes a housing 14, a drawer assembly 16, a user interface panel 20, a radiant system 22, a sterilization chamber 24, a heat sink assembly 28 and a control system 30 (FIG. 5A). As will be described herein, the sterilizing device 10 is designed to facilitate uniform light radiation from the radiant system 22 during a sterilization process that exposes medical instruments to germicidal and thermal radiation.

The housing 14 of the sterilizing device 10 is shown as generally rectangular having a front panel 14A, a rear panel 14B, a first side panel 14C a second side panel 14D, a top panel 14E and a bottom panel 14F. The respective panels 14A-14F cooperate to define a housing internal volume 34. While the exemplary sterilizing device 10 is shown as having a generally rectangular shape, other geometries are contemplated. In this regard, the sterilizing device 10 can have other geometries such as, but not limited to square, cylindrical and circular.

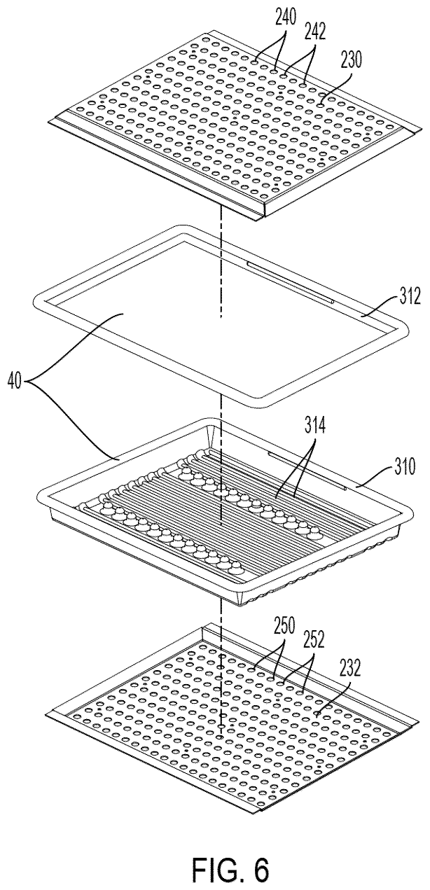

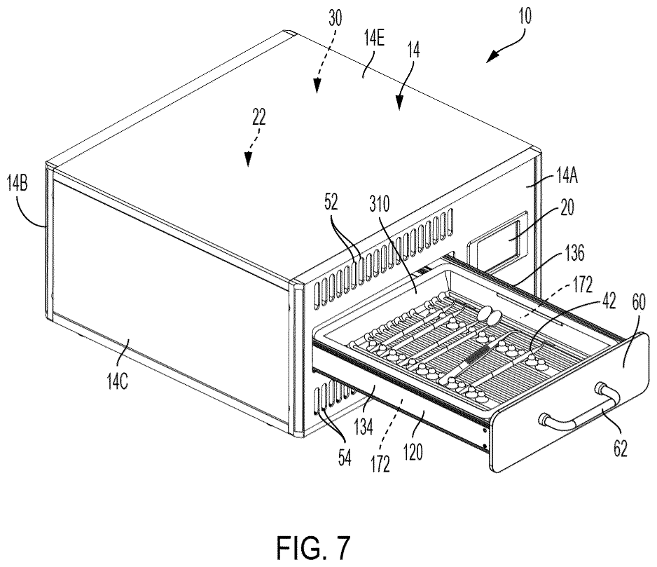

The drawer assembly 16 is configured to slidably open relative to the front panel 14A of the housing 14 from a closed position (FIG. 1) to an open position (FIG. 7). The drawer assembly 16 is configured to receive tray assemblies, such as a tray assembly 40 (FIG. 6). The tray assembly 40 is configured to hold items to be sterilized such as medical instruments, including surgical and/or dental tools 42.

The front panel 14A includes the interface panel 20, first vents 52, and second vents 54. A drawer front 60 and drawer handle 62 of the drawer assembly 16 are also provided on the front panel 14A. The bottom panel 14F includes a series of legs 66 and third vents 68. The legs 66 are configured to engage a table or other surface and offset the bottom panel 14F away from the table such that the third vents 68 can facilitate air circulation into the housing internal volume 34. The rear panel 14B includes openings 70 and 72 covered by screens 74 and 76. Fans 80 and 82 are mounted for operation on the rear panel 14B to facilitate air flow through the screens 74 and 76 to facilitate air circulation into the housing internal volume 34. A fourth vent 90 is arranged on the rear panel 14B that covers an opening 92. A power switch 94 and electrical plug interface 96 are provided on the rear panel 14B generally at the fourth vent 90.

The drawer assembly 16 will be further described. The drawer assembly 16 includes a drawer rack 110 that is supported by a rack support frame 112 that slidably traverses along drawer sliders 120 relative to a drawer frame assembly 116. In the example shown, the drawer sliders 120 have first portions fixed to the rack support frame 112 and second portions fixed to the drawer frame assembly 116. Other configurations are contemplated. The drawer frame assembly 116 includes a front drawer frame member 130, a rear drawer frame member 132, a first side drawer frame member 134 and a second side drawer frame member 136. The rack support frame 112 includes a front rack support member 142 and a rear rack support member 144. The front rack support member 142 has a first deflected mounting arm 152. The rear rack support member 144 has a second deflected mounting arm 154.

The drawer rack 110 includes a corrugated bottom support surface 160, and an outer rim 162. The outer rim 162 can collectively include a forward rim 164, a rearward rim 166, a first side rim 168 and a second side rim 170. The forward rim 164 has a forward mounting lip 180 that is coupled to the first deflected mounting arm 152 of the front rack support member 142. The rearward rim 166 has a rearward mounting lip 182 that is coupled to the second deflected mounting arm 154 of the rear rack support member 144.

An upper transparent member or window 210 and a lower transparent member or window 212 are fixedly arranged at an upper portion and lower portion of the drawer frame assembly 116. In examples, the upper and lower windows 210 and 212 can be part of the radiant system 22. The upper and lower windows 210, 212 cooperate with the drawer frame assembly 116 to define the sterilization chamber 24. In examples, the upper and lower windows 210 and 212 have transparent properties to pass UV-C and IR light emitted by the radiant system. In the examples provided, the upper and lower windows 210 and 212 can pass UV-C light having wavelengths between 200 nm and 400 nm and IR light having wavelengths between 700 nm and 1000 nm. It is appreciated that the upper and lower windows 210, 212 can be configured to pass light having other UV-C and IR wavelengths emitted from the radiant system. In examples the upper and lower windows 210 and 212 are formed of quartz. As will become appreciated herein, the sterilizing device 10 exposes the sterilizing chamber 220, and therefore the contents on the drawer rack 110, such as the contents within the tray assembly 40 with radiation during a sterilization process.

With additional reference to FIG. 6, additional features of the radiant system 22 will now be further described. The exemplary radiant system 22 shown generally includes a first radiant element or light panel 230 and a second radiant element or light panel 232. The first and second light panels 230 and 232 are positioned generally outboard of the upper and lower windows 210 and 212 respectively. In examples, the first and second light panels 230 and 232 can be similarly configured. The first light panel 230 includes a first plurality of ultraviolet-C (UV-C) light emitting diodes (LED's) 240 and a first plurality of infrared radiation (IR) LED's 242 arranged across a first printed circuit board 244. The second light panel 232 includes a second plurality of UV-C LED's 250 and a second plurality of infrared radiation IR LED's 252 arranged across a second printed circuit board 254.

In the example shown, the first plurality of UV-C LED's 240 can be alternately positioned relative to the first plurality of IR LED's 242. Similarly, the second plurality of UV-C LED's 250 can be alternately positioned relative to the second plurality of IR LED's 252. In examples, the UV-C LED's 240 and 250 can be configured to emit UV light having wavelengths between 200 nm and 400 nm. The IR LED's 242 and 252 can be configured to emit IR light having wavelengths between 700 nm and 1000 nm. It has been determined that combining radiation from both the UV range and the IR range, and more specifically the 200-400 nanometer and 700-100 nanometer ranges, provides improved sterilization results. While the exemplary radiant system 22 shown includes light panels configured with LED's, other configurations are contemplated. For example, the radiant system 22 can additionally or alternatively include UV-C lamps, IR lamps and/or heating strips.

The heat sink assembly 28 is provided in the housing internal volume 34. The heat sink assembly 28 includes a first heat sink 260 and a second heat sink 262. The heat sink assembly 28 can be configured for active or passive operation. The first heat sink 260 can be positioned generally against the first light panel 230. Similarly, the second heat sink 262 can be positioned generally against the second light panel 232. The first heat sink 260 can include a plurality of fins 264 for dissipating heat. The second heat sink 262 can include a plurality of fins 266 for dissipating heat.

The control system 30 can be housed within the housing internal volume 24 of the sterilizing device 10. The control system 30 can include one or more computing devices 270 having one or more processors, and a memory. The processor(s) can control the operation of the computing device, including implementing at least a portion of the techniques of the present disclosure. The term “processor” as used herein is intended to refer to both a single processor and multiple processors operating together, e.g. in a parallel or distributed architecture. The computing device 270 communicates with the user interface panel 20 to receive inputs from a user. It is contemplated that the user interface panel 20 can include various menu driven prompts that receive information from a user related to sterilization cycle desired and tool type included on the tray assembly 40.

The computing device 270 can further communicate with the first and second light panels 230 and 232. The computing device 270 can be configured to provide power to the light panels 230 and 232 and the fans 80 and 82. The computing device 270 can also communicate operational instructions and information with the light panels 230 and 232. In this regard, the computing device 270 can provide instructions related to powering the first and second light panels 230 and 232 “on” and “off”, and with a predetermined intensity according to a particular sterilizing schedule selected. Further, the computing device 270 can be configured to receive operational feedback from the light panels 230, 232 such as, but not limited to operational status of the LED's 240, 242, 250 and 252, temperature of the light panels 230, 232 and other statistics related to the light panels 230 and 232. It is further contemplated that the control system 30 can be configured to trigger a cooling event, such as activating the heat sink assembly 28 based on a temperature of at least one of the light panels 230, 232 exceeding a predetermined threshold.

The exemplary tray assembly 40 includes a tray 310 and a lid 312. The tray assembly 40 including the tray 310 and lid 312 is formed of fluorinated ethylene propylene (FEP). In examples, the tray 310 and lid 312 form an airtight container. The tray 310 can include extension portions 314 configured to retain respective tools and instruments. The FEP material is suitable to pass UV-C and IR light emitted by the radiant system. In other examples the lid 312 can additionally or alternatively comprise a transparent FEP film. With the tray assembly 40 is shown with a specific tool 42, on a specific configuration of the tray 310, the trays can be configured in many ways for accommodating many tools and instruments including, but not limited to, dental appliances, surgical tools, medical cart medicine drawers, mobile sterilization boxes, tattoo parlor instruments, nano-drug sterilization and paint curing applications. The tray assembly 40 is removable from the sterilizing device 10 and is customizable. In use, the drawer assembly 16 can be slid out to remove and replace the tray assembly 40 with other tray assemblies for sterilization of second set of instruments. The tray 310 can include molded-in FEP feet that offset the tray assembly 40 relative to a table on a patient preparation room increasing peace of mind for patients about their treatment.

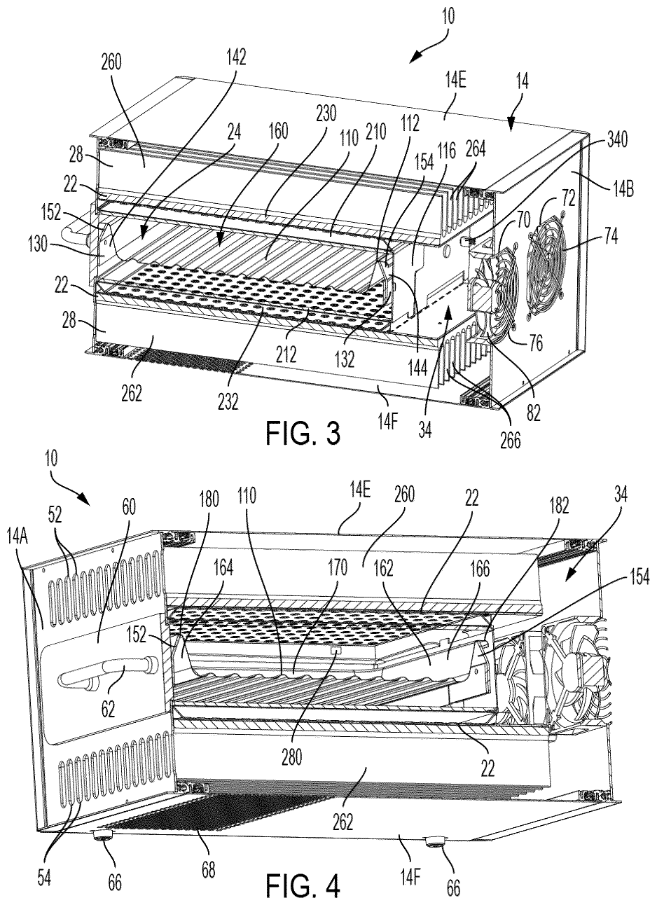

An example method of using the sterilizing device 10 will be described. At the outset a user determines which tools 42 are desired to be sterilized. In examples, the desired tools are pre-positioned in the tray assembly 40. The drawer assembly 16 is moved to the open position by grasping and pulling the handle 62 on the drawer front 60. Once the drawer assembly 40 is moved to the open position, the user places the tray assembly 40 onto the drawer rack 110. Once the tray assembly 40 is placed onto the drawer rack 110, the drawer assembly is moved to the closed position. In the closed position, the drawer assembly 16 can engage a sensor 340 (FIG. 3) that communicates a signal to the control system 30 indicative of a closed position. With confirmation of the drawer assembly 16 in the closed position, the control system 30 can permit operation of the radiant system 22 during a sterilizing process.

In examples, the user can select a desired sterilizing sequence from a menu having various digital recipes displayed on the user interface panel 50. The digital recipes can include operational parameters of the sterilization process including, but not limited to, sequence time and light intensity. The digital recipes can vary based on an identified tool type, tool material, and/or type of tray assembly. The digital recipes can be based on additional information. The sterilizing process can take a minimal amount of time to complete such as less than 10 minutes, 5 minutes, 3 minutes. Other times are contemplated. Because the sterilizing process utilizes the radiant system 22, no water is needed which reduces resource requirements and therefore cost to operate.

In other examples, the control system 30 further includes a sensing device 280. In examples, the sensing device 280 includes an optical device 280 (FIG. 4) that is configured to take an image of the contents on the tray assembly 40 and communicates it to the computing device 270. The optical device 280 can include an image sensor that is configured to output an image signal (e.g., a digital image, IR signature, a measure of reflectivity, and/or other signals) that is indicative of, or correspond to, a set of optical characteristics of a surface to be sterilized. For example only, the image signal can correspond to a measure of the reflectivity of the surface, the color of the surface, or other measure of the absorption characteristics of the radiation for the surface. The computing device 270 can determine what kind of tools are provided in the tray assembly 40 based on the image communicated by the optical device 280. In examples, the computing device can utilize an artificial intelligence, neural network, or other form of machine learning algorithm to determine the type of surface to be sterilized. In this manner, the computing device 270 can control the output of radiation from the radiant system 22 to match the type/characteristics of the surface to be sterilized to ensure an effective and efficient decontamination process. In some aspects, the image signal output by the optical device 280 can be indicative of, or correspond to, a set of optical characteristics of a pathogen present on the surface to be sterilized. For example only, the image signal can be analyzed by the computing device 270 (e.g., using a spectroscopy process) to identify an image signature corresponding to the pathogen(s) present on the surface. The image signature can be compared to a set of image signatures of known pathogens to determine a match. In this regard, the computing device 270 can retrieve a stored decontamination procedure or setting(s) corresponding to the matched pathogen. The computing device 270 can control the radiant system 22 to output radiation according to the determined decontamination procedure/setting(s) corresponding to the pathogen determined to be present on the surface. In this regard, the computing device 270 can then select a sterilizing sequence based on the tools identified by the computing device 270. It will be appreciated that different sterilizing sequences and intensities can be desirable based on particular tools.

In other examples, the sensing device 280 can include a radiation intensity sensor that is configured to sense the intensity of the UV-C radiation in the sterilizing chamber 220. The sensed intensity can be communicated to the control system 30. The control system 30 can make determinations as to whether the intensity matches a desired sterilization process and/or whether the intensity needs to be altered based on the sensed intensity and alter the intensity based on the determination.

Once the sterilizing process has been initiated, the UV-C and IR LEDs 240, 242, 250 and 252 are turned on leaving the tools 42 completely exposed to germicidal and thermal radiation. In examples, the sterilizing device 10 can successfully complete a sterilizing process using less than 100 watt-hours (Wh) of electricity. The drawer assembly 16 includes reflecting surfaces that further promote uniform radiation. The quartz windows 210 and 212 on the top and bottom of the drawer assembly 16 when the drawer assembly 16 is in the closed position provides protection to the first and second light panels 230 and 232 while letting the radiation transmit therethrough. The sterilizing device 10 is configured to only operate when the drawer assembly 16 is fully closed (such as confirmed by the sensor 340) to avoid leaking UV-C radiation that could harm the operator of the device 10. Hence, the wavelength of the UV-C LEDs 240 and 250 can be selected solely for optimization of sterilization efficacy.

While the sterilizing device 10 is shown having one sterilizing chamber 220, it may have multiple sterilizing chambers configured to enclose multiple trays each uniformly flooded with germicidal and thermal radiation. The multiple trays may have the same size or may differ in size. The tray assembly 40 may be fully removable relative to the drawer assembly 16, or it may function as one unit with the drawer assembly 16. The tray 310 may be shaped and fitted such that it serves as a tablet delivering medical or surgical tools to the treatment or operating room.

The computing device 270 is shown housed with the housing internal volume 34. The computing device 270 can alternatively be at least partially housed separately with a cable connecting the control and power unit to the sterilizing chamber 220. The exemplary sterilizing device 10 is shown as having the electrical plug interface 96 for connecting the sterilizing device 10 to alternating current power. The device may additionally or alternatively have batteries and be powered by wall power where the batteries act as backup power to complete the sterilization process in case there is a power outage.

In another embodiment, the FEP trays 310 may be fixed to the drawer and the entire drawer assembly is customized to support specific tools. The drawer is then selected and inserted into the device depending on which tool(s) must be treated. The present innovation enables uniform light radiation on any surface of a 3-dimensional object placed on the tray. The light radiation over time defined as dose is critical to deactivate any pathogens that may exist on the 3-dimensional object's surface. The two kinds of LEDs (UV-C and IR) enable simultaneous deactivation-breaking of bonds with high-energy UV-C radiation and denaturing the protein in an irreversible way with IR heating. The device provides very rapid sterilization, provides surgical tools on-demand compared to other approaches (minutes vs. hours).

The FEP tray 310 can be a dental dispensing system containing dental appliances which have gone through sterilization process. The FEP tray 310 may have multiple slots in it each receiving a respective one of the dental or surgical tools. The FEP tray 310 with the FEP cover 312 forms the tray assembly 40 that is airtight. The FEP tray 310 is designed to receive tools after ultrasonic cleaning and drying process. Such an airtight FEP tray assembly 40 (tray 310 and cover 312) may be easily placed on the drawer rack 110 of the sterilizing device 10 for sterilization treatment. After sterilization, the FEP tray assembly 40 may be brought to the operating room to be opened before dental/surgical procedure. The customizable FEP tray assembly 40 may include multiple size slots so as to accommodate different sized tools.

In examples, the UV-C and IR LEDs 240, 242, 250 and 252 can be activated concurrently during a sterilizing process. In other examples, the UV-C and IR LEDs 240, 242, 250 and 252 can be activated at different times. Due to the combination resulting from the use of the at least two separate wavelength ranges, it has been observed that the sterilizing device 10 can provide sanitization (99.9% reduction in pathogens), decontamination/disinfection (99.99% reduction in pathogens), and/or sterilization (99.9999% reduction in pathogens) in a much shorter time duration than would be expected. For example only, it has been observed that the sterilizing device 10 can provide sterilization (99.9999% reduction in pathogens) in approximately thirty to sixty seconds for some surfaces by first outputting radiation in the IR wavelength range, e.g., to raise and maintain the temperature of the surface to be between 45-185° Celsius, and then outputting radiation in the UV wavelength range. Through thermal conduction processes, the interior of the surgical instruments also reach 45-185° Celsius resulting in thermal destruction of the pathogens in occluded/shadowed areas. The combination of the energy efficiency and speed at which sterilization can be performed, resulting from the specific construction of the sterilizing device 10, provides the sterilizing device 10 with increased utility over existing sterilizing and decontamination systems.

It will be appreciated that the term “controller” as used herein refers to any suitable control device or set of multiple control devices that is/are configured to perform at least a portion of the techniques of the present disclosure. Non-limiting examples include an application-specific integrated circuit (ASIC), one or more processors and a non-transitory memory having instructions stored thereon that, when executed by the one or more processors, cause the controller to perform a set of operations corresponding to at least a portion of the techniques of the present disclosure. The one or more processors could be either a single processor or two or more processors operating in a parallel or distributed architecture.

It should be understood that the mixing and matching of features, elements, methodologies and/or functions between various examples may be expressly contemplated herein so that one skilled in the art would appreciate from the present teachings that features, elements and/or functions of one example may be incorporated into another example as appropriate, unless described otherwise above.

What is claimed is:

1. A sterilizing device for sterilizing instruments, the sterilizing device comprising:
a housing that defines an internal volume;
a drawer assembly configured to slidably open relative to the housing, the drawer assembly having a drawer rack supported by a drawer frame assembly, the drawer rack configured to receive the instruments;
a radiant system including (i) a first light panel having a first plurality of ultraviolet-C (UV-C) light emitting diodes (LED's) and a first plurality of infrared radiation (IR) LED's arranged across a first printed circuit board, the first plurality of UV-C LED's configured to emit UV-C radiation, the first plurality of IR LED's configured to emit thermal radiation, and (ii) a second light panel having a second plurality of UV-C LED's and a second plurality of IR LED's arranged across a second printed circuit board, the second plurality of UV-C LED's configured to emit UV-C radiation, the second plurality of IR LED's configured to emit thermal radiation;
a first transparent member disposed between the drawer frame assembly and the first light panel, the first transparent member and drawer frame assembly defining a sterilization chamber that receives the UV-C and thermal radiation emitted by the radiant system thereby sterilizing the instruments during a sterilizing process; and
a control system that initiates the sterilizing process based upon a user input received at a user interface panel.

2. The sterilizing device of claim 1, wherein the first light panel is arranged generally above the drawer rack and the second light panel is arranged generally below the drawer rack.

3. The sterilizing device of claim 1, further comprising a tray assembly having a tray and a cover, the tray configured to hold the instruments thereon.

4. The sterilizing device of claim 3, wherein the tray assembly is formed of fluorinated ethylene propylene (FEP) that is configured to pass UV-C and IR light emitted by the radiant system.

5. The sterilizing device of claim 1, wherein the UV-C light has wavelengths between 200 nm and 400 nm.

6. The sterilizing device of claim 5, wherein the IR light has wavelengths between 700 nm and 1000 nm.

7. The sterilizing device of claim 1, wherein the drawer rack is supported by a rack support frame, the drawer rack including a corrugated bottom surface and outer rim, the outer rim having a forward mounting lip that is coupled to a first deflected mounting arm of the rack support frame and a rearward mounting lip that is coupled to a second deflected mounting arm of the rack support frame.

8. The sterilizing device of claim 2, further comprising:
a heat sink assembly having a first heat sink positioned against the first light panel and a second heat sink positioned against the second light panel.

9. The sterilizing device of claim 1, further comprising a sensor that senses a position of the drawer assembly, wherein the sensor communicates a verification signal to the control system indicative of the drawer assembly in a closed position, the control system initiating the sterilizing process based on receipt of the verification signal.

10. The sterilizing device of claim 1, further comprising an optical device that is configured to:
capture an image of the contents on the drawer rack; and
communicate the image to the control system.

11. The sterilizing device of claim 10, wherein the control system determines an instrument type of the instruments on the drawer rack based on the captured image and determines criteria of the sterilizing process on the instrument type, the criteria including at least one of sterilization time and intensity of the emitted radiation from the LED's.

12. The sterilizing device of claim 1, further comprising a radiation intensity sensor that is configured to sense an intensity of the UV-C radiation in the sterilizing chamber and communicate the sensed intensity to the control system, the control system determining whether the intensity needs to be altered based on the sensed intensity and changing the intensity based on the determining.

13. The sterilizing device of claim 1 wherein the control system receives signals from the user interface panel indicative of criteria of the sterilizing process, the criteria including at least one of sterilization time and intensity of the emitted radiation from the LED's.

14. A method for sterilizing an instrument in a sterilizing device, the method comprising:
sliding a drawer assembly out of a housing of the sterilizing device and to an open position;
placing instruments to be sterilized onto a drawer rack of the drawer assembly;
sliding the drawer assembly back into the housing to a closed position;
determining, at a control system of the sterilizing device, a preferred sterilizing process; and
initiating, at the control system, the preferred sterilizing process, the preferred sterilizing process comprising: (i) activating a radiant system including a first light panel having a first plurality of ultraviolet-C (UV-C) light emitting diodes (LED's) and a first plurality of infrared radiation (IR) LED's arranged across a first printed circuit board, the first plurality of UV-C LED's emitting UV-C radiation, the first plurality of IR LED's emitting thermal radiation, and (ii) activating, at the radiant system, a second light panel having a second plurality of UV-C LED's and a second plurality of IR LED's arranged across a second printed circuit board, the second plurality of UV-C LED's emitting UV-C radiation, the second plurality of IR LED's emitting thermal radiation.

15. The method of claim 14, wherein emitting the UV-C light comprises emitting wavelengths between 200 nm and 400 nm.

16. The method of claim 15, wherein emitting the IR light comprises emitting wavelengths between 700 nm and 1000 nm.

17. The method of claim 16, wherein placing instruments to be sterilized onto a drawer rack comprises:
placing instruments in a tray assembly formed of fluorinated ethylene propylene (FEP) that is configured to pass UV-C and IR light emitted by the radiant system.

18. The method of claim 16, wherein determining a preferred sterilizing process comprises:
capturing, at an optical device disposed in the sterilizing device, an image of the instruments;
communicating the image to the control system;
determining, at the control system, a type of instruments in the sterilizing device based on the image; and
selecting criteria of the preferred sterilizing process based on the type of instruments, the criteria including at least one of sterilization time and intensity of the emitted radiation from the LED's.

19. The method of claim 16, wherein determining a preferred sterilizing process comprises:
receiving, at the control system, a selected sterilizing process input by a user at the user interface panel.

20. A sterilizing device for sterilizing instruments, the sterilizing device comprising:
a housing that defines an internal volume;
a drawer assembly configured to slidably open relative to the housing, the drawer assembly having a drawer rack supported by a drawer frame assembly, the drawer rack configured to receive the instruments;
a radiant system including a first light panel having a first plurality of ultraviolet-C (UV-C) light emitting diodes (LED's) and a first plurality of infrared radiation (IR) LED's arranged across a first printed circuit board, the first plurality of UV-C LED's configured to emit UV-C radiation, the first plurality of IR LED's configured to emit thermal radiation;
a first transparent member disposed between the drawer frame assembly and the first light panel, the first transparent member and drawer frame assembly defining a sterilization chamber that receives the UV-C and thermal radiation emitted by the radiant system thereby sterilizing the instruments during a sterilizing process;
a tray assembly having a tray and a cover, the tray configured to hold the instruments thereon, wherein the tray assembly is formed of fluorinated ethylene propylene (FEP) that is configured to pass UV-C and IR light emitted by the radiant system; and
a control system that initiates the sterilizing process based upon a user input received at a user interface panel.
